(19) United States
(12) Patent Application Publication
(10) Pub. No.: US 2024/0238373 A1
(43) Pub. Date: Jul. 18, 2024

(54) Use of Erythropoietin (EPO) or Erythropoiesis-Stimulating Agent (ESA) to Treat Osteomyelitis, Bone Infection, and Bone Inflammation

(71) Applicant: Eyal Attias, Jerusalem (IL)

(72) Inventor: Eyal Attias, Jerusalem (IL)

(21) Appl. No.: 18/290,297

(22) PCT Filed: May 16, 2022
(86) PCT No.: PCT/IL2022/050509

§ 371 Date: Nov. 12, 2023

Related U.S. Application Data
(60) Provisional application No. 63/189,721, filed on May 18, 2021.

(51) Int. Cl. A61K 38/18; A61K 45/06; A61P 19/00

(52) U.S. Cl. CPC A61K 38/1816; A61K 45/06; A61P 19/00

(57) ABSTRACT

Erythropoietin or Erythropoiesis-Stimulating Agent (ESA) for use in treating bone inflammation or bone infection or Osteomyelitis. An effective dosage of Erythropoietin or ESA is administered to a human subject via: subcutaneous injection, intravenous injection, subcutaneous administration, intravenous administration, subcutaneous implant, oral delivery, or a combination thereof. The dosage is in a range of 3,000 to 10,000 units per administration session, or 4,000 to 6,000 units per administration session. The administration frequency is 2 or 3 times per week, for a period of at least 6 weeks. Optionally, an antibiotic agent is also administered, concurrently or prior to or after the administration of the Erythropoietin or ESA. Particularly, the human subject is non-Anemic, and is not or was not diagnosed with Anemia, and did not or does not suffer from Anemia. Optionally, the administration of Erythropoietin or EP A may prevent limp amputation, particularly for a diabetic subject.

### CROSS-REFERENCE TO RELATED APPLICATIONS

This patent application claims priority and benefit from U.S. 63/189,721, filed on May 18, 2021, which is hereby incorporated by reference in its entirety.

### FIELD

Some embodiments are related to the field of medicine and pharmaceutical products.

### BACKGROUND

Osteomyelitis (OM) is infection or inflammation of the bone, such as due to bacterial infection or fungal infection. It may occur, for example, by spread from the blood or from the tissue surrounding the bone. Some symptoms of Osteomyelitis may include, for example: pain in a specific bone with overlying redness, fever, and weakness.

### SUMMARY

In accordance with some embodiments, Erythropoietin or an Erythropoiesis-Stimulating Agent (ESA) may be used or administered or injected (e.g., subcutaneously, and/or via other delivery methods) to a patient having Osteomyelitis or having bone infection or having bone inflammation or having inflammation of the bone; and particularly to a patient having Osteomyelitis or bone infection and not currently diagnosed with (and/or treated for) Anemia; in order to treat and/or cure and/or mitigate Osteomyelitis or bone infection or bone inflammation or inflammation of the bone, and/or to mitigate or reduce or alleviate Osteomyelitis or Osteomyelitis pain or Osteomyelitis symptoms, and/or in order to otherwise improve quality-of-life of a person having Osteomyelitis or bone infection or bone inflammation or inflammation of the bone, and particularly of a person who does not have Anemia and/or who was not or is not currently diagnosed with Anemia.

Erythropoietin or Erythropoiesis-Stimulating Agent (ESA) for use in treating bone inflammation or bone infection or Osteomyelitis. An effective dosage of Erythropoietin or ESA is administered to a human subject via: subcutaneous injection, intravenous injection, subcutaneous administration, intravenous administration, subcutaneous implant, oral delivery, or a combination thereof. The dosage is in a range of 3,000 to 10,000 units per administration session, or 4,000 to 6,000 units per administration session. The administration frequency is 2 or 3 times per week; for example, every other day, or every three days; for a period of at least 6 consecutive weeks. Optionally, an antibiotic agent is also administered, concurrently or prior to or after the administration of the Erythropoietin or ESA. Particularly, the human subject is non-Anemic, and is not or was not diagnosed with Anemia, and did not or does not suffer from Anemia. In some embodiments, the administration of Erythropoietin or EPA may prevent limp amputation, particularly for a diabetic subject.

Some embodiments may provide other and/or additional advantages and/or benefits.

### BRIEF DESCRIPTION OF THE DRAWINGS

FIG. 1 is a flow-chart of a method, in accordance with some demonstrative embodiments.

### DETAILED DESCRIPTION OF SOME DEMONSTRATIVE EMBODIMENTS

The Applicants have realized that Erythropoietin (EPO) is a hormone produced by the kidney; that Erythropoietin helps the bone marrow produce red blood cells; and that man-made Erythropoiesis-Stimulating Agents (ESAs) are currently indicated for treatment of Anemia.

The term “Erythropoietin” as used herein may include, for example: the hormone known as Erythropoietin (EPO); or, an artificial or man-made or synthetic or recombinant version of Erythropoietin; or, an artificial or synthetic or man-made or recombinant Erythropoiesis-Stimulating Agent (ESA), or recombinant Erythropoietin, or recombinant human Erythropoietin (rhEPO), EPO-type substances, or recombinant epoetin; or, an artificial or man-made or synthetic epoetin, such as artificial or man-made or synthetic epoetin alfa or epoetin alpha or epoetin-α, epoetin beta or epoetin-β, epoetin gamma or epoetin-γ, epoetin delta or epoetin-δ, epoetin omega or epoetin-ω; or other type of ESA that stimulates the bone marrow (and/or other body organs or body parts) to produce more red blood cell or to increase the production of red blood cell or to hasten or expedite or trigger the production of red blood cells; or a combination or mixture of two or more of the above-mentioned materials.

The term “treating” as used herein includes, for example, ameliorating, mitigating, alleviating, and/or reducing the instances of a disease or condition, or the symptoms of a disease or condition, or the negative outcomes or negative effects of having a disease or condition, or the human pain or human suffering due to a disease or condition; and/or improving or increasing or enhancing the quality-of-life of a person having a disease or condition.

The Applicants have discovered and realized that administration of (such as subcutaneous injection of, or subcutaneous administration of) Erythropoietin or EPO or rhEPO or an ESA (such as epoetin, epoetin alpha, epoetin beta, epoetin gamma, epoetin delta, epoetin omega), in accordance with a particular effective dosage or a particular effective amount, or in accordance with a therapeutically effective amount or a therapeutically effective dosage, in accordance with a particular administration protocol and/or administration frequency, may treat or cure or mitigate Osteomyelitis or bone infection or boney inflammation (e.g., bone inflammation that may be caused by an infection, bacterial bone infection, fungal bone infection); even though for many years, Erythropoietin or ESA or epoetin are and have been indicated for the treatment of Anemia due to Chronic Kidney Disease (CKD); and even though there were no previous attempts or suggestions or indications to administer or to subcutaneously administer Erythropoietin or ESA or epoetin for the treatment of Osteomyelitis or bone infection or bone inflammation.

The Applicants have realized and discovered, that administering or subcutaneously injecting or subcutaneously administering Erythropoietin or ESA to a patient having Osteomyelitis or to a patient diagnosed with Osteomyelitis, may treat the Osteomyelitis and/or may cure the Osteomyelitis, and/or may remove or reduce or cure bone infection or bone inflammation, and/or may remove or extinguish some of (or most of, or a majority of, or all of) the bone infection or the bone inflammation, and/or may reduce or mitigate or alleviate symptoms or pain associated with Osteomyelitis or bone infection or bone inflammation.

In accordance with some embodiments, Erythropoietin or ESA is administered or is given or is subcutaneously injected or is subcutaneously administered to a patient having Osteomyelitis or having bone inflammation or bone infection, wherein the patient does not have Anemia, and/or wherein the patient is not currently diagnosed with Anemia, and/or wherein the patient has never been diagnosed with Anemia, and/or wherein the patient does not currently exhibit and/or did not previously exhibit symptoms of Anemia, and/or wherein the patient currently has normal or within-the-norm value of hemoglobin in the blood, and/or wherein the patient is a person who has Anemia but is not treated (regularly, or at all) with Erythropoietin or ESA.

In accordance with some embodiments, Erythropoietin or ESA may be administered subcutaneously to an Osteomyelitis patient; for example, via a subcutaneous injection or shot or jab, or via an under-the-skin injection or shot or jab, or via an injector device (e.g., a one-time injector device or syringe, or a refillable or reusable injector device or syringe). In some embodiments, the Erythropoietin or ESA may be administered in one or more other and/or additional and/or alternate ways, for example, via oral drug delivery, via intravenous (IV) drug delivery, via an implanted device that performs regulated release and/or slow release and/or gradual release and/or time-controlled release, and/or via other suitable delivery methods or devices.

In accordance with some embodiments, the effective dosage or the effective amount or the effective quantity or the therapeutically effective amount or the therapeutically effective dosage of Erythropoietin or ESA, that may be administered or given or may be subcutaneously administered to an Osteomyelitis patient in order to treat Osteomyelitis, may be, for example (e.g., per subcutaneous shot, or per subcutaneous injection, or per an administration session, or per an administration event): 2,000 units or International Units (IUs), or 2,500 units or IUs, or 3,000 units or IUs, or 3,500 units or IUs, or 4,000 units or IUs, or 4,500 units or IUs, or 5,000 units or IUs, or 5,500 units or IUs, or 6,000 units or IUs, or 6,500 units or IUs, or 7,000 units or IUs, or 7,000 units or IUs, or 7,500 units or IUs, or 8,000 units or IUs, or 8,500 units or IUs, or 9,000 units or IUs, or 9,500 units or IUs, or 10,000 units or IUs.

In some embodiments, the effective dosage or the effective amount or the effective quantity or the therapeutically effective amount or the therapeutically effective dosage of Erythropoietin or ESA, that may be administered or given or may be subcutaneously administered to an Osteomyelitis patient in order to treat Osteomyelitis, may be, for example (e.g., per subcutaneous shot, or per subcutaneous injection, or per an administration session, or per an administration event): in the range of 2,000 to 10,000 units or IUs; or in the range of 2,500 to 10,000 units or IUs; or in the range of 3,000 to 10,000 units or IUs; or in the range of 3,500 to 10,000 units or IUs; or in the range of 4,000 to 10,000 units or IUs; or in the range of 4,500 to 10,000 units or IUs; or in the range of 5,000 to 10,000 units or IUs; or in the range of 5,500 to 10,000 units or IUs; or in the range of 6,000 to 10,000 units or IUs; or in the range of 6,500 to 10,000 units or IUs; or in the range of 7,000 to 10,000 units or IUs; or in the range of 2,000 to 9,000 units or IUs; or in the range of 2,500 to 9,000 units or IUs; or in the range of 3,000 to 9,000 units or IUs; or in the range of 3,500 to 9,000 units or IUs; or in the range of 4,000 to 9,000 units or IUs; or in the range of 4,500 to 9,000 units or IUs; or in the range of 5,000 to 9,000 units or IUs; or in the range of 5,500 to 9,000 units or IUs; or in the range of 6,000 to 9,000 units or IUs; or in the range of 6,500 to 9,000 units or IUs; or in the range of 7,000 to 9,000 units or IUs; or in the range of 2,000 to 8,000 units or IUs; or in the range of 2,500 to 8,000 units or IUs; or in the range of 3,000 to 8,000 units or IUs; or in the range of 3,500 to 8,000 units or IUs; or in the range of 4,000 to 8,000 units or IUs; or in the range of 4,500 to 8,000 units or IUs; or in the range of 5,000 to 8,000 units or IUs; or in the range of 5,500 to 8,000 units or IUs; or in the range of 6,000 to 8,000 units or IUs; or in the range of 6,500 to 8,000 units or IUs; or in the range of 7,000 to 8,000 units or IUs; or in the range of 2,000 to 7,000 units or IUs; or in the range of 2,500 to 7,000 units or IUs; or in the range of 3,000 to 7,000 units or IUs; or in the range of 3,500 to 7,000 units or IUs; or in the range of 4,000 to 7,000 units or IUs; or in the range of 4,500 to 7,000 units or IUs; or in the range of 5,000 to 7,000 units or IUs; or in the range of 5,500 to 7,000 units or IUs; or in the range of 6,000 to 7,000 units or IUs; or in the range of 6,500 to 7,000 units or IUs; or in the range of 6,500 to 7,500 units or IUs; or in the range of 2,000 to 6,500 units or IUs; or in the range of 2,500 to 6,500 units or IUs; or in the range of 3,000 to 6,500 units or IUs; or in the range of 3,500 to 6,500 units or IUs; or in the range of 4,000 to 6,500 units or IUs; or in the range of 4,500 to 6,500 units or IUs; or in the range of 5,000 to 6,500 units or IUs; or in the range of 5,500 to 6,500 units or IUs; or in the range of 2,000 to 6,000 units or IUs; or in the range of 2,500 to 6,000 units or IUs; or in the range of 3,000 to 6,000 units or IUs; or in the range of 3,500 to 6,000 units or IUs; or in the range of 4,000 to 6,000 units or IUs; or in the range of 4,500 to 6,000 units or IUs; or in the range of 5,000 to 6,000 units or IUs; or in the range of 5,500 to 6,000 units or IUs; or in the range of 2,000 to 5,500 units or IUs; or in the range of 2,500 to 5,500 units or IUs; or in the range of 3,000 to 5,500 units or IUs; or in the range of 3,500 to 5,500 units or IUs; or in the range of 4,000 to 5,500 units or IUs; or in the range of 4,500 to 5,500 units or IUs; or in the range of 5,000 to 5,500 units or IUs; or in the range of 2,000 to 5,000 units or IUs; or in the range of 2,500 to 5,000 units or IUs; or in the range of 3,000 to 5,000 units or IUs; or in the range of 3,500 to 5,000 units or IUs; or in the range of 4,000 to 5,000 units or IUs; or in the range of 4,500 to 5,000 units or IUs; or in the range of 3,000 to 3,500 units or IUs; or in the range of 3,000 to 4,000 units or IUs; or in the range of 3,500 to 4,000 units or IUs; or in the range of 3,500 to 4,500 units or IUs; or in the range of 4,000 to 5,000 units or IUs; or in the range of 4,500 to 5,000 units or IUs.

In some embodiments, the effective dosage or the effective amount or the effective quantity or the therapeutically effective amount or the therapeutically effective dosage of Erythropoietin or ESA, that may be administered or given or may be subcutaneously administered to an Osteomyelitis patient in order to treat Osteomyelitis, may be, for example (e.g., per subcutaneous shot, or per subcutaneous injection, or per an administration session, or per an administration event): 0.50 or 0.75 or 1.00 or 1.25 or 1.50 or 1.75 or 2.00 milliliters of ESA or Epoetin or of Epoetin alfa; and/or may be ESA or Epoetin or of Epoetin alfa at a concentration of 3,000 or 3,500 or 4,000 or 4,500 or 6,000 units or IUs of per milliliter of injectable solution; and/or may be ESA or Epoetin or Epoetin alfa at am amount of 20 or 22 or 24 or 25 or 28 or 30 or 32 or 33 or 33.5 or 34 or 35 or 36 or 38 or 40 or 45 or 50 micrograms; and/or may be ESA or Epoetin or Epoetin alfa at an amount in the range of 20 to 50 micrograms, or at an amount in the range of 20 to 45 micrograms, or at an amount in the range of 20 to 40 micrograms, or at an amount in the range of 20 to 35 micrograms, or at an amount in the range of 20 to 30 micrograms, or at an amount in the range of 20 to 25 micrograms, or at an amount in the range of 25 to 50 micrograms, or at an amount in the range of 30 to 50 micrograms, or at an amount in the range of 35 to 50 micrograms, or at an amount in the range of 40 to 50 micrograms, or at an amount in the range of 45 to 50 micrograms, or at an amount in the range of 25 to 45 micrograms, or at an amount in the range of 25 to 40 micrograms, or at an amount in the range of 30 to 45 micrograms, or at an amount in the range of 30 to 45 micrograms, or at an amount in the range of 30 to 36 micrograms, or at an amount in the range of 30 to 35 micrograms, or at an amount in the range of 32 to 36 micrograms.

In some embodiments, the effective dosage or the effective amount or the effective quantity or the therapeutically effective amount or the therapeutically effective dosage of Erythropoietin or ESA, may be a function of (or dependent on) the weight of the body of the patient; for example, 50 or 60 or 70 or 80 or 90 or 100 or 120 or 140 or 150 units or IUs of Erythropoietin or ESA per kilogram of body-weight; or 50 to 150 units or IUs of Erythropoietin or ESA per kilogram of body-weight; or 75 to 150 units or IUs of Erythropoietin or ESA per kilogram of body-weight; or 100 to 150 units or IUs of Erythropoietin or ESA per kilogram of body-weight; or 75 to 125 units or IUs of Erythropoietin or ESA per kilogram of body-weight; or 100 to 125 units or IUs of Erythropoietin or ESA per kilogram of body-weight; or 50 to 100 units or IUs of Erythropoietin or ESA per kilogram of body-weight; or 50 to 125 units or IUs of Erythropoietin or ESA per kilogram of body-weight; or 50 to 75 units or IUs of Erythropoietin or ESA per kilogram of body-weight; or 75 to 125 units or IUs of Erythropoietin or ESA per kilogram of body-weight; or 75 to 125 units or IUs of Erythropoietin or ESA per kilogram of body-weight.

In some embodiments, the frequency of injecting or administering or subcutaneously administering or subcutaneously injecting the effective dosage or the therapeutically effective amount or the therapeutically effective dosage of Erythropoietin or ESA to an Osteomyelitis patient in order to treat Osteomyelitis, may be: once a week; once per 7 consecutive days; twice per week; twice per 7 consecutive days; three times per week; three times per 7 consecutive days; four times per week; four times per 7 consecutive days; five times a week; five times per 7 consecutive days; once per day; two times per day; three times per day; twice per month; every other week; once per 10 consecutive days; once per 14 consecutive days.

In some embodiments, the time-length for treating an Osteomyelitis patient by administration or by subcutaneous administration of a therapeutically effective dosage of Erythropoietin or ESA may be: one week; or two weeks; or three weeks; or four weeks; or five weeks; or six weeks; or eight weeks.

In some embodiments, two consecutive administration or subcutaneous administration sessions of Erythropoietin or ESA to the same Osteomyelitis patient (e.g., performed at least one day apart, or performed at least 6 hours apart) may have different dosages or effective quantities or effective amounts; for example, administering or subcutaneously administering 4,000 units or IUs of Erythropoietin or ESA on Monday, then administering or subcutaneously administering 5,000 units or IUs of Erythropoietin or ESA on Wednesday, then administering or subcutaneously administering 6,000 units or IUs of Erythropoietin or ESA on Friday, and then repeating that order (or, a different order) of differential dosages over several weeks.

In some embodiments, for example, a first administration or subcutaneous administration session delivers to the patient an amount A1 of micrograms or units or IUs of Erythropoietin or ESA; then, a second administration or subcutaneous administration session delivers to the patient an amount A2 of micrograms or units or IUs of Erythropoietin or ESA; wherein A1<A2. Optionally, those two administration sessions are performed within a particular time-period (e.g., one week); and are then repeated for N more time-intervals or time-periods (e.g., for N more weeks; in each week, repeating again the effective dosage of A1 and then A2).

In some embodiments, for example, a first administration or subcutaneous administration session delivers to the patient an amount B1 of micrograms or units or IUs of Erythropoietin or ESA; then, a second administration or subcutaneous administration session delivers to the patient an amount B2 of micrograms or units or IUs of Erythropoietin or ESA; wherein B1>B2. Optionally, those two administration sessions are performed within a particular time-period (e.g., one week); and are then repeated for N more time-intervals or time-periods (e.g., for N more weeks; in each week, repeating again the effective dosage of B1 and then B2).

In some embodiments, for example, a first administration or subcutaneous administration session delivers to the patient an amount C1 of micrograms or units or IUs of Erythropoietin or ESA; then, a second administration or subcutaneous administration session delivers to the patient an amount C2 of micrograms or units or IUs of Erythropoietin or ESA; then, a third administration or subcutaneous administration session delivers to the patient an amount C3 of micrograms or units or IUs of Erythropoietin or ESA; wherein C1<C2 and wherein C2<C3. Optionally, those three administration sessions are then repeated (e.g., every week, for N weeks).

In some embodiments, for example, a first administration or subcutaneous administration session delivers to the patient an amount D1 of micrograms or units or IUs of Erythropoietin or ESA; then, a second administration or subcutaneous administration session delivers to the patient an amount D2 of micrograms or units or IUs of Erythropoietin or ESA; then, a third administration or subcutaneous administration session delivers to the patient an amount D3 of micrograms or units or IUs of Erythropoietin or ESA; wherein C1>C2 and wherein C2>C3. Optionally, those three administration sessions are then repeated (e.g., every week, for N weeks).

In some embodiments, the Erythropoietin or ESA may be produced, for example, in (or by using) Chinese Hamster Ovary (CHO) cells by recombinant DNA technology.

In some embodiments, the Erythropoietin or ESA that is administered or is subcutaneously administered to an Osteomyelitis patient may be or may include, for example: an injectable solution known as “EPREX” injectable solution (available from Janssen-Cilag Ltd) which is used and is indicated for treatment of Anemia; or an injectable solution known as “EPOGEN” injectable solution (available from Amgen) which is used and is indicated for treatment of Anemia; or an injectable solution known as “PROCRIT” injectable solution (available from Janssen) which is used and is indicated for treatment of Anemia; or an injectable solution known as “RETACRIT” injectable solution (available from Pfizer) which is used and is indicated for treatment of Anemia; and/or other suitable epoetin or epoetin alfa injectable solution; or a biosimilar equivalent of epoetin alfa; or am injectable solution of epoetin alfa-epbx.

In accordance with some embodiments, an injectable solution may comprise: as an active ingredient, epoetin alfa or epoetin alfa-epbx; and as inactive ingredients: albumin (human), citric acid, sodium chloride, sodium citrate, and Water for Injection; and optionally (e.g., in a multiple-dose vial) also benzyl alcohol.

In accordance with some embodiments, an injectable solution may comprise: as an active ingredient, epoetin alfa or epoetin alfa-epbx; and as inactive ingredients: Polysorbate 80, sodium chloride, sodium dihydrogen phosphate, dihydrate, disodium phosphate dihydrate, glycine, and water for injections.

In accordance with some embodiments, a single-use injectable solution or a single-dose vial may comprise: as an active ingredient, epoetin alfa or epoetin alfa-epbx; and as inactive ingredients: calcium chloride dehydrate, glycine, isoleucine, leucine, L-glutamic acid, phenylalanine, polysorbate 20, sodium chloride, sodium phosphate dibasic anhydrous, sodium phosphate monobasic monohydrate, and threonine, in water for injection. Optionally, sodium hydroxide and hydrochloric acid may be added to adjust the pH. Optionally, benzyl alcohol may be added to multiple-dose vials. For example, a multiple-dose vial of injectable solution may comprise epoetin alfa or epoetin alfa-epbx, with the following inactive ingredients: benzyl alcohol, L-methionine, polysorbate 20, sodium phosphate dibasic anhydrous, sodium phosphate monobasic monohydrate, and sucrose, in water for injection. Sodium hydroxide and hydrochloric acid may be added to adjust the pH.

In some embodiments, a single-dose vial may be formulated with an isotonic sodium chloride/sodium citrate-buffered solution, and may be produced or supplied in multiple different strengths; for example, each single-dose one-milliliter vial of injectable solution may comprise 2,000 or 3,000 or 4,000 or 5,000 or 6,000 or 10,000 Units or International Units (IUs) of epoetin alfa or epoetin alfa-epbx. Additionally, it may comprise inactive ingredients; for example: Albumin (Human) (2.5 mg), citric acid (0.06 mg), sodium chloride (5.9 mg), and sodium citrate (5.8 mg) in Water for Injection, USP (pH 6.9±0.3).

In some embodiments, a two-milliliter vial of injectable solution may comprise as active ingredient 10,000 units or IUs of epoetin alfa or epoetin alfa-epbx; as well as inactive ingredients, for example: albumin (human) (2.5 mg), benzyl alcohol (1%), sodium chloride (8.2 mg), citric acid (0.11 mg), and sodium citrate (1.3 mg) per 1 mL Water for Injection, USP (pH 6.1±0.3).

In some embodiments, instead of subcutaneously injecting the effective dosage of Erythropoietin or ESA to an Osteomyelitis patient via a syringe, or in addition to such subcutaneous injection, the effective dosage of Erythropoietin or ESA may be subcutaneously provided or subcutaneously administered to Osteomyelitis patient via other drug delivery means; for example, by subcutaneously implanting or providing a device that performs controlled release and/or gradual release of Erythropoietin or ESA under the skin of the Osteomyelitis patient.

The Applicants have realized that administering or giving or subcutaneously injecting the effective dosage or the therapeutically effective amount of Erythropoietin or ESA to an Osteomyelitis patient may increase or stimulate the production of Red Blood Cells (RBCs), which in turn may increase the mechanical and/or physical pressure or force that is applied onto some bones or bone-portions, thereby causing removal or exit of bacteria from the infected bone(s); or, may cause other effects or results within the patient's body which may lead, directly or indirectly, to removal or mitigation of the bone infection or the bone inflammation or the Osteomyelitis. The Applicants note that there may be other bio-chemical reasons or bio-physical reasons that cause removal or exit of bacteria from an infected bone due to subcutaneously injecting the effective dosage of Erythropoietin or ESA to an Osteomyelitis patient.

In some embodiments, administering or subcutaneously administering, or injecting or subcutaneously injecting, the effective dosage or the therapeutically effective amount of Erythropoietin or ESA to an Osteomyelitis patient may prevent amputation of a limb or arm or leg of the patient. In some embodiments, the treatment of subcutaneously injecting the effective dosage of Erythropoietin or ESA to an Osteomyelitis patient may be accompanied by also administering to the patient one or more antibiotic drugs, orally and/or via intravenous (IV) means, based on the particular type of bacteria that may be detected in the bone(s) and/or based on possible allergy or sensitivity of a patient to particular antibiotic agent(s) and/or based on detection of possible resistance of the bone bacteria to a particular antibiotic agent.

In some embodiments, due to the subcutaneous injection or administration of the effective dosage of Erythropoietin or ESA to an Osteomyelitis patient, a reduced amount of antibiotics may be required, and/or a reduce time-length (e.g., in days, or in weeks) of antibiotics administration may be required, and/or a weaker type of antibiotics may be used, and/or (in some situations, or for some patients) no antibiotics at all may be needed in order to combat and treat the bone infection.

In accordance with some embodiments, Erythropoietin or ESA may be administered to an Osteomyelitis patient via subcutaneous injection or subcutaneous administration. In some embodiments, additionally or alternatively, Erythropoietin or ESA may be administered to an Osteomyelitis patient via one or more other drug delivery methods; for example, via non-subcutaneous drug delivery method(s) and/or via intravenous drug delivery method(s) and/or via an under-the-skin implantation method(s) and/or via oral drug delivery method(s) and/or via in-the-vain drug delivery and/or via in-the-vein injection and/or via other drug delivery method(s).

In accordance with some embodiments, improvement in the bone infection may be monitored and/or detected and/or determined based on one or more indicators; for example, an improvement in the general clinical condition of the patient, an improvement in one or more particular clinical conditions of the patient, reduced pain, reduced weakness, reduced fatigue, increased strength, improvement in the ability of the patient to move and/or utilize arms and/or legs and/or limbs and/or body organs that had bone infection, increased appetite, improvement in an appearance of a wound or injury that led to the bone infection, reduction of swelling, reduction in amount of pus at or near the infected bone or body part, improvement in the closure or healing of an injury or wound that led to the bone infection (e.g., may be observed visually), strengthening of the infected bone structure, fusion or patching of a bone fracture or a bone portion that was infected (e.g., may be detected by touching or feeling, or by X-ray), physical or spatial improvements to the infected bone portions (e.g., detected via X-ray or ultrasound imaging), reduction in infection or inflammation indicators or attributes (e.g., reduction in fever, reduction in white blood cell count, reduction in neutrophils, reduction in Erythrocyte Sedimentation Rate (ESR), reduction in C-Reactive Protein (CRP), reduction in Plasma Viscosity (PV), and/or other indicators), improvement in metabolic indicators (e.g., increase in hemoglobin, increase in albumin).

In accordance with some embodiments, administering or subcutaneously injecting or subcutaneously administering the effective dosage or the therapeutically effective amount of Erythropoietin or ESA to an Osteomyelitis patient may be performed even in the presence of a foreign object that is neighboring the bone infection; for example, in the presence of an implanted screw or connector or wire or supporting element, made of titanium or metal or ceramic or bio-friendly plastic or polyethylene or cross-linked polyethylene; or in the presence of artificial or man-made or implanted elements that are used for hip replacement, knee replacement, shoulder replacement, or other treatments; or in the presence of other foreign object such as a gun bullet or a glass portion that is embedded in or near the bone and cannot be efficiently removed. In accordance with some embodiments, removal of the foreign object is preferred; however, if such removal is difficult or inefficient or dangerous or impossible, the subcutaneous injection or administration of the effective dosage of Erythropoietin or ESA to an Osteomyelitis patient may still be performed. Optionally, cleaning and/or sanitizing and/or washing of the injured area and/or a nearby channel or sinuses or fistula may be performed, to improve the treatment results in some patients.

In accordance with some embodiments, the administration or the injection or the subcutaneous injection or the subcutaneous administration of the effective dosage or the therapeutically effective amount of Erythropoietin or ESA to an Osteomyelitis patient may be performed even if an initial test indicates that the patient appears to have a sufficient or adequate level of Erythropoietin; particularly if the patient is also diagnosed with (or is suspected to have) Anemia, and/of if the patient currently has an insufficient level of hemoglobin (e.g., less than 12 or 12.3 or 12.5 grams per deciliter for females; less than 13.0 or 13.5 grams per deciliter for males; other threshold values may be used, optionally taking into account the patient's age and/or other characteristics). In accordance with some embodiments, the administration or the injection or the subcutaneous injection or the subcutaneous administration of the effective dosage or the therapeutically effective amount of Erythropoietin or ESA to such an Osteomyelitis patient may still assist in (or contribute to) the treatment of Osteomyelitis, and/or may hasten the recovery from Osteomyelitis, and/or may shorten the time-length required for Osteomyelitis treatment, and/or may reduce the frequency and/or strength of antibiotics that are administered to the patient, and/or may reduce the probability that an amputation would be required, and/or may otherwise support or assist or improve the treatment of Osteomyelitis for such patient.

In accordance with some embodiments, the administration or the injection or the subcutaneous injection or the subcutaneous administration of the effective dosage or the therapeutically effective amount of Erythropoietin or ESA to an Osteomyelitis patient may be stopped (or, may be paused) when or if one or more pre-defined conditions hold true; for example: the symptoms of Osteomyelitis have disappeared; the symptoms of Osteomyelitis have significantly decreased; the level of hemoglobin have increased to a normal level or has increased beyond a pre-defined threshold value; the wound or injury that is associated with the bone infection has recovered, and/or an associated bone fracture has fused or has remodeled; a particular protocol for subcutaneous injection or administration of the effective dosage of Erythropoietin or ESA to such as Osteomyelitis patient has ended (e.g., after N administration sessions, or after M weeks of treatment; wherein N and M are pre-defined values).

For demonstrative purposes, some embodiments are described in the context of administering Erythropoietin or ESA to a human in order to treat bone infection or bone inflammation or Osteomyelitis. However, some embodiments may similarly include administering Erythropoietin or ESA to a mammal or to a non-human animal having bone infection or bacterial bone infection or fungal bone infection or bone inflammation or bacterial bone inflammation or fungal bone inflammation; for example, a dog, a cat, a canine animal, a feline animal, a cow, a horse, a pet, or other mammals or animals.

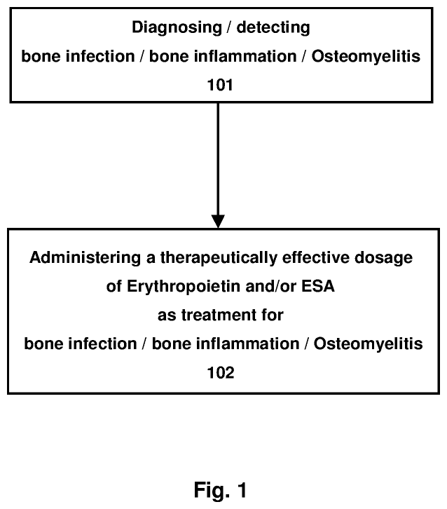

Reference is made to FIG. 1, which is a flow-chart of a method in accordance with some demonstrative embodiments. The method may include, for example: diagnosing or detecting (or, in some embodiments, suspecting) bone infection or bone inflammation or Osteomyelitis of a subject (block 101); and administering to that subject a therapeutically effective dosage of Erythropoietin or ESA as treatment for bone infection or bone inflammation or Osteomyelitis (block 102).

In some embodiments, the subject is non-Anemic, and does not have and/or did not have Anemia, and/or was not diagnosed with Anemia, and/or is not currently diagnosed with Anemia, and/or did not exhibit (and/or does not currently exhibit) symptoms or negative effects of Anemia.

Some embodiments include: Erythropoietin for use in treating Osteomyelitis.

Some embodiments include: Erythropoietin for use in treating bone infection or bone inflammation.

Some embodiments include: Erythropoiesis-Stimulating Agent (ESA) for use in treating Osteomyelitis.

Some embodiments include: Erythropoiesis-Stimulating Agent (ESA) for use in treating bone infection or bone inflammation.

Some embodiments include: Erythropoietin or Erythropoiesis-Stimulating Agent (ESA), for use in treating Osteomyelitis or bone infection or bone inflammation.

Some embodiments include: Erythropoietin or ESA for use in accordance with any of the above, wherein the Erythropoietin or ESA is configured to be administered at an effective dosage or at a therapeutically effective amount to a person having Osteomyelitis; via subcutaneous injection and/or via other subcutaneous administration method and/or via non-subcutaneous drug delivery method and/or via intravenous drug delivery method and/or via an under-the-skin implant and/or via oral drug delivery and/or via in-the-vain drug delivery and/or via in-the-vein injection and/or via other drug delivery method.

Some embodiments include: Erythropoietin or ESA for use in accordance with any of the above, wherein the effective dosage or the therapeutically effective amount is in a range of 3,000 to 10,000 units of ESA per administration session.

Some embodiments include: Erythropoietin or ESA for use in accordance with any of the above, wherein the effective dosage or the therapeutically effective amount is in a range of 4,000 to 6,000 units of ESA per administration session.

Some embodiments include: Erythropoietin or ESA for use in accordance with any of the above, further accompanied by administration of an antibiotic agent via oral antibiotic drug delivery and/or via intravenous antibiotic drug delivery.

Some embodiments include: Use of Erythropoietin or Erythropoiesis-Stimulating Agent (ESA) to treat Osteomyelitis.

Some embodiments include: the use of Erythropoietin or ESA to treat Osteomyelitis in accordance with any of the above, by administering Erythropoietin or ESA at an effective dosage or at a therapeutically effective amount to a person having Osteomyelitis, via subcutaneous injection or via other subcutaneous administration method or via non-subcutaneous drug delivery means.

Some embodiments include: the use of Erythropoietin or ESA to treat Osteomyelitis in accordance with any of the above, wherein the effective dosage or the therapeutically effective amount is in a range of 3,000 to 10,000 units of ESA per administration session.

Some embodiments include: the use of Erythropoietin or ESA to treat Osteomyelitis in accordance with any of the above, wherein the effective dosage or the therapeutically effective amount is in a range of 4,000 to 6,000 units of ESA per administration session.

Some embodiments include: the use of Erythropoietin or ESA to treat Osteomyelitis in accordance with any of the above, further accompanied by administration of an antibiotic agent via oral antibiotic drug delivery and/or via intravenous antibiotic drug delivery.

Some embodiments include: a medicament for treating or mitigating or curing Osteomyelitis, the medicament comprising: an effective dosage or a therapeutically effective amount of Erythropoietin or Erythropoiesis-Stimulating Agent (ESA) in a subcutaneously injectable medium. In some embodiments, the effective dosage or the therapeutically effective amount is in a range of 3,000 to 10,000 units of ESA per administration session. In some embodiments, the effective dosage or the therapeutically effective amount is in a range of 4,000 to 6,000 units of ESA per administration session.

Some embodiments include: a medicament for treating or mitigating or curing Osteomyelitis, the medicament comprising: an effective dosage or a therapeutically effective amount of Erythropoietin or Erythropoiesis-Stimulating Agent (ESA) in a medium suitable for oral administration to a person. In some embodiments, the effective dosage or the therapeutically effective amount is in a range of 3,000 to 10,000 units of ESA per administration session. In some embodiments, the effective dosage or the therapeutically effective amount is in a range of 4,000 to 6,000 units of ESA per administration session.

Some embodiments include: a medicament for treating or mitigating or curing Osteomyelitis, the medicament comprising: an effective dosage or a therapeutically effective amount of Erythropoietin or Erythropoiesis-Stimulating Agent (ESA) in a medium suitable for intravenous administration to a person. In some embodiments, the effective dosage or the therapeutically effective amount is in a range of 3,000 to 10,000 units of ESA per administration session. In some embodiments, the effective dosage or the therapeutically effective amount is in a range of 4,000 to 6,000 units of ESA per administration session.

Some embodiments include an apparatus useful in treatment or curing or mitigation of Osteomyelitis, the apparatus comprising: a single-use syringe, storing a subcutaneously injectable medium which comprises an effective dosage or a therapeutically effective amount of Erythropoietin or Erythropoiesis-Stimulating Agent (ESA) for an Osteomyelitis patient.

Some embodiments include an apparatus useful in treatment or curing or mitigation of Osteomyelitis, the apparatus comprising: a vial storing a subcutaneously injectable medium which comprises an effective dosage or a therapeutically effective amount of Erythropoietin or Erythropoiesis-Stimulating Agent (ESA) for an Osteomyelitis patient.

Some embodiments include a method of treating or mitigating or curing Osteomyelitis, the method comprising: subcutaneously administering to an Osteomyelitis patient, an effective dosage or a therapeutically effective amount of Erythropoietin or Erythropoiesis-Stimulating Agent (ESA), for treating or mitigating or curing Osteomyelitis.

In some embodiments, the method includes: administering, not subcutaneously or non-subcutaneously, to an Osteomyelitis patient, an effective dosage or a therapeutically effective amount of Erythropoietin or Erythropoiesis-Stimulating Agent (ESA), for treating or mitigating or curing Osteomyelitis

Some embodiments include a method for manufacturing a medicament for treating or mitigating or curing Osteomyelitis, the method comprising: manufacturing an injectable medium which comprises an effective dosage or a therapeutically effective amount of Erythropoietin or Erythropoiesis-Stimulating Agent (ESA) for an Osteomyelitis patient.

Some embodiments include a system for manufacturing a medicament for treating or mitigating or curing Osteomyelitis, the system comprising: a production unit configured to produce an injectable medium which comprises an effective dosage or a therapeutically effective amount of Erythropoietin or Erythropoiesis-Stimulating Agent (ESA) for an Osteomyelitis patient.

Some embodiments include: The use of Erythropoietin or ESA to treat Osteomyelitis of an Osteomyelitis patient who is not diagnosed with Anemia or who does not currently have Anemia; by administering Erythropoietin or ESA at an effective dosage or at a therapeutically effective amount to a person having Osteomyelitis, via subcutaneous injection or via other subcutaneous administration method or via non-subcutaneous administration method.

Some embodiments include: Erythropoietin or Erythropoiesis-Stimulating Agent (ESA), at an effective dosage or at a therapeutically effective amount for use in treating Osteomyelitis or bone infection of a person who does not have Anemia and/or who is not currently diagnosed with Anemia and/or who was not diagnosed with Anemia.

Some embodiments provide: Erythropoietin or Erythropoiesis-Stimulating Agent (ESA) or Recombinant Human EPO (rhEPO), for use in treating a bone inflammation or bone infection or Osteomyelitis. According to some embodiments, the bone inflammation is caused by an infection. According to some embodiments, the bone inflammation is Osteomyelitis.

According to some embodiments, the Erythropoietin or ESA is for administering by an administration route selected from the group consisting of: subcutaneous injection, subcutaneous administration, intravenous (IV) injection, intravenous (IV) administration, subcutaneous implant, oral delivery, and combinations of two or more of said administration routes. According to some embodiments, the Erythropoietin or ESA is for administering by subcutaneous injection.

According to some embodiments, the Erythropoietin or ESA is for administering at a dosage in a range of 3,000 to 10,000 units per administration session.

According to some embodiments, the Erythropoietin or ESA is for administering at a dosage in a range of 4,000 to 6,000 units per administration session.

According to some embodiments, the Erythropoietin or ESA is for administering at a frequency of from twice to three times per week, for a period of at least 6 weeks; or for a period of 6 or 8 or 10 or 12 weeks. In some embodiments, administration sessions are performed three times per week, on alternating days; for example, on Monday and Wednesday and Friday of every week, for a period of 6 or 8 or 10 or 12 consecutive weeks. In some embodiments, administration sessions are performed two times per week; for example, on Monday and Thursday of every week, or on Tuesday and Friday of every week; for a period of 6 or 8 or 10 or 12 consecutive weeks. In some embodiments, administration sessions are performed three times per week, on alternating days; for example, on Sunday and Tuesday and Thursday of every week, for a period of 6 or 8 or 10 or 12 consecutive weeks. In some embodiments, the period of time may be extended to 16 or 20 or 24 or 26 weeks; for example, if CT scan and/or MRI and/or bone scan indicate a gradual or partial improvement (namely, reduction or decrease of the bone inflammation or bone infection; or reduction in the risk or threat of limp amputation).

According to some embodiments, the Erythropoietin or ESA is for co-administration with an antibiotic agent; for example, for an entirety of a time period in which the antibiotic agent is administered, and in parallel (temporally) with such administration of the antibiotic agent. In some embodiments, the antibiotic agent is administered together with (or concurrently with, or at least partially concurrently with, or simultaneously with, or at least partially simultaneously with) the Erythropoietin or ESA. In some embodiments, additionally or alternatively, the antibiotic agent is administered within H1 hours prior to the administration of the Erythropoietin or ESA; wherein H1 is, for example, 1 or 2 or 3 or 4 or 6 or 12 or 18 or 24 hours. In some embodiments, additionally or alternatively, the antibiotic agent is administered within H2 subsequently to the administration of the Erythropoietin or ESA; wherein H2 is, for example, 1 or 2 or 3 or 4 or 6 or 12 or 18 or 24 hours.

According to some embodiments, the Erythropoietin or ESA and the antibiotic agent are for administering in a single dosage form; or as a single mixture, or as a single mixed solution.

According to some embodiments, the Erythropoietin or ESA and the antibiotic agent are for administering in separate dosage forms, wherein the administering is performed independently, sequentially, simultaneously or concomitantly, or any combination thereof. According to some embodiments, the Erythropoietin or ESA is for administering prior to administration of the antibiotic agent. According to some embodiments, the Erythropoietin or ESA is for administering subsequent to administration of the antibiotic agent.

According to some embodiments, the antibiotic agent is for administration orally or by subcutaneous injection.

In some embodiments, the administration of the Erythropoietin or ESA may be performed concurrently with, or in addition to, surgical intervention or surgical procedures or other medical procedures, that may assist in parallel to mitigating or treating the bone infection or bone inflammation or Osteomyelitis; and/or prior to surgical intervention or surgical procedures or other medical procedures; and or subsequent to such surgical intervention or surgical procedures or other medical procedures; which may include, for example, surgical or other medical intervention for draining or removing at least some of the materials that are at the location of the bone infection or bone inflammation or Osteomyelitis.

According to some embodiments, the treating is of a non-anemic subject, or of a human that is not and/or was not diagnosed with Anemia, or of a person who does not present or exhibit Anemia symptoms, or of a person who does not regularly or sporadically (or at all) taking Anemia drugs or Anemia-related drugs or Anemia-treating drugs. In some embodiments, the subject is a Diabetic (and non-anemic) subject, who may be facing a risk or threat of partial or complete limp amputation or body-part amputation or body-organ amputation due to the bone infection or bone inflammation or Osteomyelitis; and the treatment with the therapeutically effective amount of Erythropoietin or Erythropoiesis-Stimulating Agent (ESA) may reduce or decrease, or even remove, such risk or threat.

According to a further aspect of some embodiments disclosed herein, there is provided a method for treating a bone inflammation in a subject in need thereof, the method comprising administering to the subject a therapeutically effective amount of Erythropoietin or Erythropoiesis-Stimulating Agent (ESA). According to some embodiments of the method, the bone inflammation is caused by an infection. According to some embodiments of the method, the bone inflammation is Osteomyelitis.

According to some embodiments of the method, the administering is by a route selected from the group consisting of: subcutaneous injection, subcutaneous administration, intravenous (IV) injection, intravenous (IV) administration, subcutaneous implant, oral delivery, and combinations of two or more of said administration routes. According to some embodiments of the method, the route comprises subcutaneous injection.

According to some embodiments of the method, the therapeutically effective amount is in a range of 3,000 to 10,000 units per administration session.

According to some embodiments of the method, the therapeutically effective amount is in a range of 4,000 to 6,000 units per administration session.

According to some embodiments of the method, the administering is conducted at a frequency of from twice to three times per week.

According to some embodiments, the method further comprises administering an antibiotic agent.

According to some embodiments of the method, the Erythropoietin or ESA and the antibiotic agent are administered in a single dosage form.

According to some embodiments of the method, the Erythropoietin or ESA and the antibiotic agent are administered in separate dosage forms, wherein the administering is performed independently, sequentially, simultaneously or concomitantly, or any combination thereof. According to some embodiments of the method, the Erythropoietin or ESA is administered prior to administration of the antibiotic agent. According to some embodiments, the Erythropoietin or ESA is administered subsequent to administration of the antibiotic agent.

According to some embodiments of the method, the antibiotic is administered orally or by subcutaneous injection.

According to some embodiments of the method, the subject is a non-anemic subject.

Functions, operations, components and/or features described herein with reference to one or more embodiments, may be combined with, or may be utilized in combination with, one or more other functions, operations, components and/or features described herein with reference to one or more other embodiments, or vice versa.

While certain features of some embodiments have been illustrated and described herein, many modifications, substitutions, changes, and equivalents may occur to those skilled in the art. Accordingly, the claims are intended to cover all such modifications, substitutions, changes, and equivalents.

### EXAMPLES

### Example 1

An adult male subject is diagnosed as suffering from Osteomyelitis, such as by magnetic resonance imaging (MRI). The subject is treated twice weekly with 5,000 International Units (IU) of erythropoietin (or ESA) per administration session, via subcutaneous injection, for a period of 6 or 8 or 10 weeks. At the end of the treatment period, a statistically significant decrease in Osteomyelitis is identified by a subsequent MRI.

### Example 2

A juvenile female subject is diagnosed as suffering from Osteomyelitis, such as by computed topography (CT) scanning. The subject is treated three times per week by oral administration of 3,000 International Units (IU) of erythropoietin (or ESA) per administration session for a period of three months. At the end of the treatment period, a statistically significant decrease in Osteomyelitis is identified by a subsequent CT scan.

### Example 3

An adult female subject is diagnosed as suffering from Osteomyelitis, such as by MRI or CT scan. The subject is treated twice weekly by oral administration of 6,000 International Units (IU) of epoetin alpha per administration session, followed immediately by oral administration of an antibiotic agent, for a period of two months. At the end of the treatment period, a statistically significant decrease in Osteomyelitis is identified by a subsequent MRI or CT scan.

### Example 4

A juvenile male subject is diagnosed as suffering from Osteomyelitis, such as by CT scan or MRI. The subject is treated on alternate days by oral administration of an antibiotic agent, followed by intravenous injection of 8,000 International Units of epoetin alpha (or epoetin beta, or other type of epoetin or ESA) per administration session, followed immediately by oral administration of an antibiotic agent, for a period of four months. At the end of the treatment period, a statistically significant decrease in Osteomyelitis is identified by a subsequent CT scan or MRI.

### Example 5

An adult diabetic male subject is diagnosed as suffering from Osteomyelitis of Diabetic foot, such as by Clinical physical examination and/or bone scan and/or by CT scan and/or via MRI. This subject is under threat of limb amputation, due to this Osteomyelitis. The subject is treated on alternate days by intravenous administration of an antibiotic agent, followed by subcutaneous injection of 8,000 International Units of epoetin alpha (or epoetin beta, or other type of epoetin or ESA) per administration session, followed immediately by oral administration of an antibiotic agent, for a period of three months. At the end of the treatment period, a statistically significant decrease in Osteomyelitis is identified by a subsequent Clinical physical examination and/or bone scan and/or by CT scan and/or MRI, and the threat of limb amputation has decreased.

1. Erythropoietin or Erythropoiesis-Stimulating Agent (ESA) for use in treating a bone inflammation.

2. Erythropoietin or ESA for use in accordance with claim 1,
wherein said bone inflammation is caused by an infection.

3. Erythropoietin or ESA for use in accordance with claim 1,
wherein said bone inflammation is Osteomyelitis.

4. Erythropoietin or ESA for use in accordance with claim 1,
for administering by an administration route selected from the group consisting of:
subcutaneous injection,
intravenous injection,
subcutaneous administration,
intravenous administration,
subcutaneous implant,
oral delivery, and
a combination of two or more of the above-listed administration routes.

5. Erythropoietin or ESA for use in accordance with claim 4,
for administering by subcutaneous injection.

6. Erythropoietin or ESA for use in accordance with claim 1,
for administering at a dosage in a range of 3,000 to 10,000 units per administration session.

7. Erythropoietin or ESA for use in accordance with claim 1,
for administering at a dosage in a range of 4,000 to 6,000 units per administration session.

8. Erythropoietin or ESA for use in accordance with claim 7,
for administering at a frequency of from two times to three times per week, for a period of at least six weeks.

9. Erythropoietin or ESA for use in accordance with claim 1,
for co-administration with an antibiotic agent.

10. Erythropoietin or ESA for use in accordance with claim 9,
wherein said antibiotic agent is for administration orally or by subcutaneous injection.

11. Erythropoietin or ESA for use in accordance with claim 1,
wherein said treating is of a non-anemic subject.

12. A method for treating a bone inflammation in a subject in need thereof,
the method comprising:
administering to the subject a therapeutically effective amount of Erythropoietin or Erythropoiesis-Stimulating Agent (ESA).

13. The method of claim 12,
wherein the bone inflammation is caused by an infection.

14. The method of claim 12,
wherein the bone inflammation is Osteomyelitis.

15. The method of claim 12,
wherein said administering is by an administration route selected from the group consisting of:
subcutaneous injection,
intravenous injection,
subcutaneous administration,
intravenous administration,
subcutaneous implant,
oral delivery, and
a combination of two or more of the above-listed administration routes

16. The method of claim 15,
wherein said administration route comprises subcutaneous injection.

17. The method of claim 12,
wherein said therapeutically effective amount is in a range of 3,000 to 10,000 units per administration session.

18. The method of claim 17,
wherein said therapeutically effective amount is in a range of 4,000 to 6,000 units per administration session.

19. The method of claim 12,
wherein said administering is conducted at a frequency of from two times to three times per week.

20. The method of claim 12,
further comprising: administering to said subject an antibiotic agent.

21. The method of claim 20,
wherein said antibiotic is administered orally or by subcutaneous injection.

22. The method of claim 12,
wherein said subject is a non-anemic subject.
